(19) United States
(12) Patent Application Publication
(10) Pub. No.: US 2023/0357711 A1
(43) Pub. Date: Nov. 9, 2023

(54) CELL SUPPORT AND METHOD OF PRODUCING SAME, CELL CULTURE METHOD AND CELL STRUCTURE

(71) Applicant: Konica Minolta, Inc., Tokyo (JP)

(72) Inventors: TEMMEI ITO, Sagamihara-shi, Kanagawa (JP); AKIHIRO MAEZAWA, Hino-shi, Tokyo (JP); YASUHIKO TABATA, Uji-shi, Kyoto (JP); YUKI MURATA, Kyoto-shi, Kyoto (JP)

(21) Appl. No.: 18/026,474

(22) PCT Filed: Mar. 23, 2021
(86) PCT No.: PCT/JP2021/011903

§ 371 Date: Mar. 15, 2023

(30) Foreign Application Priority Data
Sep. 17, 2020 (WO) PCT/JP2020/035197

(51) Int. Cl. C12N 5/00

(52) U.S. Cl. CPC C12N 5/0068; C12N 2533/30; C12N 2529/00; C12N 2533/54

(57) ABSTRACT

The present invention relates to a cell support including a base material containing a biocompatible substance or a base material having a surface to which the biocompatible substance is imparted, and gelatin particles held by the biocompatible substance of the base material. The cell support can be produced by a method of causing the base material containing a biocompatible substance to hold the gelatin particles. By seeding cells on the cell support and culturing the cells, a cell structure in which a reagent or a drug has been uniformly taken into more cells can be obtained.

### TECHNICAL FIELD

The present invention relates to a cell support, a method of producing the same, a cell culture method, and a cell structure.

### BACKGROUND ART

A technique for introducing a reagent, a drug, or the like into a cultured cell is known. For example, Patent Literature 1 describes that a probe solution is added to a culture solution, the probe is introduced into a cell, and a state of the cell can be detected.

In addition, a support for culturing cells for transplantation or holding and storing cells (hereinafter, also simply referred to as “cell support”) is known. For example, Patent Literature 2 describes a biodegradable base material including a biodegradable nonwoven fabric sewn by a biodegradable filament, and a biodegradable film-like material superimposed on the biodegradable nonwoven fabric. Patent Literature 2 describes that when the biodegradable base material is used as a cell support, cells easily enter the biodegradable base material and can be embedded therein for a long period of time without causing a foreign substance reaction in a living body.

Patent Literature 3 describes that cells are cultured by coating a poly N-isopropylacrylamide (PIPAAm) culture dish (UpCell manufactured by Cellseed Co., Ltd.) with gelatin or the like. Patent Literature 4 describes that adhesive cancer cells are cultured using a cell culture base material in which a biobinding polymer layer is formed on a substrate made of a material such as silicon, glass, or plastic. Patent Literature 5 describes that cells are cultured with a gelatin nonwoven fabric.

### CITATION LIST

### Patent Literature

Patent Literature 1: JP 2016-077159 A
Patent Literature 2: JP 2004-148014 A
Patent Literature 3: WO 2014/192909 A
Patent Literature 4: WO 2018/182044 A
Patent Literature 5: WO 2018/235745 A

### SUMMARY OF INVENTION

### Technical Problem

As described in Patent Literature 1, when a reagent, a drug, or the like is to be introduced into a cell, there is a problem that a deviation occurs in uptake of the reagent, the drug, or the like into the cell. In particular, when a colony of cells is to be grown in a three-dimensional direction, a deviation is likely to occur in uptake of the reagent, the drug, or the like, and cells that have taken these and cells that have not taken these are generated, or cells that have taken a large amount of these and cells that have taken only a small amount of these are generated. This deviation in uptake of cells also occurs when cells are cultured using a base material simply using a biocompatible substance on a surface in contact with the cells as described in Patent Literatures 2 to 5.

The present invention has been made based on the above findings, and an object thereof is to provide a cell support and a method of producing the same, a cell culture method using the cell support, and a cell structure produced using the cell support, capable of more uniformly introducing a reagent, a drug, or the like into a cell.

### Solution to Problem

The above problem is solved by a cell support including a base material containing a biocompatible substance or a base material having a surface to which the biocompatible substance is imparted, and gelatin particles held by a surface of the base material in contact with the cells.

In addition, the above problem is solved by a cell culture method including a step of preparing the cell support and a step of seeding cells on the cell support.

In addition, the above problem is solved by a cell structure including the cell support and cells held by the cell support.

### Advantageous Effects of Invention

The present invention provides a cell support and a method of producing the same, a cell culture method using the cell support, and a cell structure produced using the cell support, capable of more uniformly introducing a reagent, a drug, or the like into a cell.

### BRIEF DESCRIPTION OF DRAWINGS

FIGS. 1A and 1B are schematic diagrams illustrating a configuration of a cell support according to an embodiment of the present invention.

FIG. 2 is a fluorescence image observed in Experiment 1.

FIG. 3 is a fluorescence image observed in Experiment 2.

FIG. 4A is a fluorescent image observed from a cell support using gelatin nonwoven fabric 1 in Experiment 3. FIG. 4B is a fluorescent image observed from a cell support using gelatin nonwoven fabric 2 in Experiment 3. FIG. 4C is a fluorescent image observed from a cell support using a polypropylene nonwoven fabric coated with gelatin in Experiment 3. FIG. 4D is a fluorescent image observed from a cell support using a polypropylene nonwoven fabric not coated with gelatin in Experiment 3.

### DESCRIPTION OF EMBODIMENTS

Hereinafter, an embodiment of the present invention will be described in detail with reference to the drawings. Note that the present invention is not limited to the following embodiment.

[Cell Support]

A cell support according to the embodiment of the present invention includes a base material containing a biocompatible substance or a base material having a surface to which the biocompatible substance is imparted, and gelatin particles held by the base material.

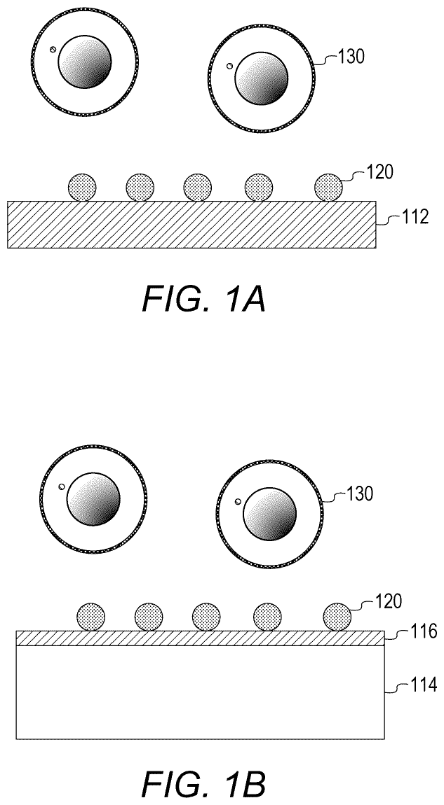

FIGS. 1A and 1B are schematic diagrams illustrating a configuration of the cell support according to the present embodiment. In the example illustrated in FIG. 1A, a cell support 100 includes a base material 112 containing a biocompatible substance and gelatin particles 120 held by the biocompatible substance of the base material 112. In the example illustrated in FIG. 1B, the cell support 100 includes a base material 114 such as glass or plastic, a biocompatible substance 116 imparted to a surface of the base material, and the gelatin particles 120 held by the biocompatible substance 116. These cell supports 100 can be used as a support for culturing seeded cells 130 or holding and storing the seeded cells 130.

(Base Material)

The base material only needs to be a base material capable of culturing and holding cells, and may be a base material having a two-dimensional shape for growing cells into a planar colony, or may be a base material having a three-dimensional shape for growing cells into a three-dimensional colony.

Examples of the base material having a two-dimensional shape include a cell culture plate, a culture dish, and a Petri dish. In addition, the base material having a two-dimensional shape may have a shape such as a bottle shape, a tube shape, a bag shape, a microchannel shape, or a multiwell-plate shape.

Examples of the base material having a three-dimensional shape include an assembly of fibers, such as a nonwoven fabric, a woven fabric, or a net, and a porous base material such as a membrane filter or a mesh sheet.

Materials of these base materials are not particularly limited, and may be any material such as metal, resin, glass, or ceramic.

Examples of the metal include titanium, nickel, platinum, gold, tungsten, iron, and an alloy thereof.

Examples of the resin include: a polyolefin including polyurethane, polyethylene, polypropylene, and the like; polycarbonate, polylactic acid, polyglycolic acid, poly-ε-caprolactone, polyvinyl alcohol, and a copolymer thereof; a synthetic resin including polyethylene glycol, polyimide, an acrylic resin, polyester, polyvinylidene fluoride, polyvinyl alcohol, polyvinyl chloride, polyvinyl acetate, polyvinyl pyrrolidone, polystyrene, a copolymer thereof, and the like; a natural resin such as cellulose; an elastomer including a styrene-butadiene copolymer, polyisoprene, an isobutylene-isoprene copolymer (butyl rubber), a halogenated butyl rubber, a butadiene-styrene-acrylonitrile copolymer, a silicon polymer, a fluorosilicon polymer, and the like; and a material derived from a living body, such as polyhydroxybutyric acid, polyhydroxyvaleric acid, protein, sugar, or glycoprotein (fibronectin or the like).

The base material may be a scaffold material that mimics an extracellular matrix for growing cells into a three-dimensional colony to form a spheroid. Examples of the scaffold material include a gel-like body of a biocompatible substance described later, including gelatin, collagen, hyaluronic acid, and the like.

(Biocompatible Substance)

The base material contains a biocompatible substance on a surface thereof.

The form of the base material is not particularly limited as long as the base material contains a biocompatible substance on a surface thereof. Abase material having the above shape may be formed of a biocompatible substance, or a biocompatible substance may be imparted to a surface of the base material formed into a predetermined shape.

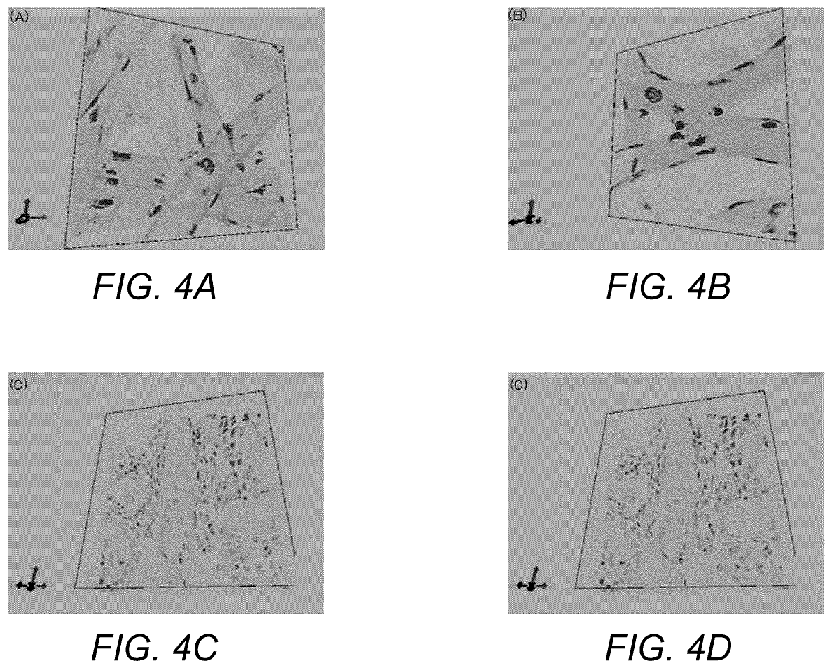

According to findings of the present inventors, in a configuration in which gelatin particles are simply imparted to a base material such as silicon, glass, or plastic, an efficiency of uptake of the gelatin particles into cells is not so high. Meanwhile, when the base material contains a biocompatible substance and the biocompatible substance holds gelatin particles, an efficiency of uptake of the gelatin particles into cells is remarkably increased (see comparison between FIGS. 4A to 4C and FIG. 4D, and comparison between cell supports 3 to 5 and cell supports 1 and 2 in Table 3).

Note that when the base material is a porous base material or the like having a three-dimensional shape, a biocompatible substance does not need to be imparted to an inner surface of the base material, but only needs to be imparted to at least an outer surface of the base material on which cells are seeded, and may be imparted only to the outer surface of the base material on which cells are seeded.

The biocompatible substance may be a polymer derived from a living body or a biodegradable synthetic polymer.

Examples of the polymer derived from a living body include: a biodegradable polyester such as polyhydroxybutyric acid or polyhydroxyvaleric acid; a glycosaminoglycan (such as hyaluronic acid), starch, cellulose or a derivative thereof (such as carboxymethylcellulose); a polysaccharide such as alginic acid, chitin, or chitosan; a poly(amino acid) such as collagen, elastin, gelatin, or laminin; a glycoprotein such as fibronectin, and a complex thereof. Among these, collagen, gelatin, fibronectin, laminin, and a polysaccharide are preferable.

Examples of the biodegradable synthetic polymer include polylactic acid, polyglycolic acid, poly-ε-caprolactone, polyvinyl alcohol, a copolymer thereof, polyethylene glycol, polyhydroxybutyric acid, polyhydroxyvaleric acid, and a biodegradable polyester. Among these, polylactic acid, polyglycolic acid, poly-ε-caprolactone, polyvinyl alcohol, and a copolymer thereof are preferable.

According to findings of the present inventors, among these biocompatible substances, a polymer material having a higher water content is likely to increase and control an introduction ratio of gelatin particles into cells. The biocompatible substance is preferably collagen, gelatin, fibronectin, a polyvinyl alcohol or a copolymer thereof, chitin, or chitosan, and more preferably collagen, gelatin, fibronectin, or polyvinyl alcohol from the above viewpoint.

In addition, among these biocompatible substances, a poly(amino acid) is preferable, and gelatin is more preferable because of having high affinity for gelatin particles and easily controlling an introduction ratio of gelatin particles into cells.

The gelatin may be any known gelatin obtained by modifying collagen derived from bovine bone, cow skin, pig skin, pig tendon, fish scales, fish meat, and the like.

The gelatin may be crosslinked. The crosslinking may be crosslinking with a crosslinking agent, or may be self-crosslinking performed without using a crosslinking agent.

The crosslinking agent only needs to be a compound having a plurality of functional groups that forms a chemical bond with, for example, a hydroxy group, a carboxyl group, an amino group, a thiol group, or an imidazole group. Examples of such a crosslinking agent include: glutaraldehyde; a water-soluble carbodiimide including 1-ethyl-3-(3-dimethylaminopropyl) carbodiimide hydrochloride (EDC) and 1-cyclohexyl-3-(2-morpholinoethyl) carbodiimide-metho-p-toluenesulfonate (CMC); a compound having two or more epoxy groups, including ethylene glycol diglycidyl ether, polyethylene glycol diglycidyl ether, polyglycerol polyglycidyl ether, and glycerol polyglycidyl ether; and propylene oxide. Among these, glutaraldehyde and EDC are preferable, and glutaraldehyde is more preferable from a viewpoint of further enhancing reactivity.

Examples of the self-crosslinking include crosslinking by application of heat or irradiation with an electron beam or an ultraviolet ray.

In the present embodiment, an introduction ratio of gelatin particles into cells to be seeded and grown can be controlled depending on the type of biocompatible substance. For example, when it is desired to increase the introduction ratio of gelatin particles into cells, it is preferable to use a biocompatible substance closer to gelatin, or to make the degree of crosslinking of gelatin as a biocompatible substance closer to the degree of crosslinking of gelatin in gelatin particles. Alternatively, when it is desired to suppress the introduction ratio of gelatin particles into cells to some extent, it is preferable to use a biocompatible substance farther from gelatin, or to make the degree of crosslinking of gelatin as a biocompatible substance farther from the degree of crosslinking of gelatin in gelatin particles. Thus, it is desirable to select the type of biocompatible substance according to the introduction ratio of the gelatin particles to be achieved.

Note that a difference in the degree of crosslinking of the gelatin can be estimated from a difference in signal intensity of any energy band in a loss spectrum detected by transmission electron microscope observation (TEM-EELS measurement) in combination with electron energy-loss spectroscopy, a peak intensity ratio between COOH and CONH (peak intensity of COOH/peak intensity of CONH) in a spectrum obtained by plotting a wave number on the horizontal axis and an absorbance on the vertical axis, obtained by measurement with a Fourier transform type infrared spectrophotometer (FT-IR), or the like.

By controlling the introduction ratio of the gelatin particles, an introduction ratio of a reagent or a drug carried by the gelatin particles into cells can also be controlled. The amount to be introduced into cells also varies depending on the type of reagent or drug. Therefore, the introduction ratio of the gelatin particles into cells is controlled by the biocompatible substance, whereby the introduction ratio of the reagent or the drug into the cells is also controlled, whereby the amount of the reagent or the drug to be introduced into the cells can be easily controlled.

(Gelatin Particles)

The base material holds gelatin particles.

The gelatin particles are in contact with the biocompatible substance of the base material and are immobilized on the base material.

At this time, the gelatin particles are immobilized and held at positions in contact with the biocompatible substance contained in the base material. For example, when the biocompatible substance is imparted to a surface of the base material, the gelatin particles are imparted to and immobilized on a region of the base material to which the biocompatible substance is imparted.

In addition, when the base material is a porous base material or the like having a three-dimensional shape, the gelatin particles do not need to be imparted to an inner surface of the base material, but only need to be imparted to at least an outer surface of the base material on which cells are seeded, and may be imparted only to the outer surface of the base material on which cells are seeded.

The gelatin particles may be nanoparticles made of any known gelatin similar to those described for the biocompatible substance. Gelatin has been used for food and medical purposes for a long time, and is less harmful to a human body even when being ingested into the body. In addition, since gelatin is dispersed and lost in a living body, there is an advantage that it is not necessary to remove the gelatin from the living body.

Gelatin constituting the gelatin particles preferably has a weight average molecular weight of 1000 or more and 100000 or less. The weight average molecular weight can be, for example, a value measured in accordance with PAGI method, tenth edition (2006).

Gelatin constituting the gelatin particles may be crosslinked. The crosslinking may be crosslinking with the above-described crosslinking agent, or may be self-crosslinking performed without using a crosslinking agent.

The gelatin particles are preferably cationized, for example, by introduction of a primary amino group, a secondary amino group, a tertiary amino group, or a quaternary ammonium group from a viewpoint of easily controlling ease of uptake of the gelatin particles into cells.

The gelatin particles can be cationized by a known method of introducing a functional group to be cationized under physiological conditions during production. For example, by causing a reaction of an alkyldiamine including ethylenediamine, N,N-dimethyl-1,3-diaminopropane, and the like, trimethylammonium acetohydrazide, spermine, spermidine, a diethylamide chloride, and the like using a condensing agent including 1-ethyl-3-(3-dimethylaminopropyl) carbodiimide hydrochloride, cyanuric chloride, N,N′-carbodiimidazole, cyanogen bromide, a diepoxy compound, tosyl chloride, a dianhydride compound such as diethyltriamine-N,N,N′,N″,N″-pentanoic acid dianhydride, tricyl chloride, and the like, the amino group can be introduced into a hydroxy group or a carboxyl group of gelatin.

The gelatin particles preferably have an average particle size of 100 nm or more and 1000 nm or less. Although the gelatin particles carry a probe, the gelatin particles do not substantially have a probe in a surface layer portion thereof. Therefore, even when the gelatin particles have an average particle size of 1000 nm or less, the gelatin particles are easily taken into cells by the cells' own activity. In order to allow many gelatin particles to be taken into cells in a shorter time, the average particle size of the gelatin particles is more preferably 800 nm or less. Meanwhile, the gelatin particles having an average particle size of 100 nm or more easily carry a probe in the particles, and can increase a capacity of the probe. The larger the average particle size of the gelatin particles is within a range of 1000 nm or less, the more easily the gelatin particles are taken into cells by the cells' own activity. The average particle size of the gelatin particles is preferably 200 nm or more, and more preferably 300 nm or more from the above viewpoint.

Note that the average particle size of the gelatin particles can be an apparent particle size of the gelatin particles measured by a dynamic light scattering method. Alternatively, the average particle size of the gelatin particles can be a value obtained by averaging a major axis and a minor axis. The minor axis and the major axis of the gelatin particles can be values obtained by analyzing an image obtained by imaging the dried gelatin particles after the gelatin particles are allowed to stand in the atmosphere at 80° C. for 24 hours with a scanning electron microscope (SEM). Since the gelatin particles are usually in a form of an assembly formed of a plurality of gelatin particles, the major axis, the minor axis, and the particle size of the gelatin particle can be values obtained by averaging the major axis, the minor axis, and the particle size of a plurality of gelatin particles (for example, 20 gelatin particles) arbitrarily selected from the assembly, respectively. When there is a difference between the average particle sizes measured by these methods, it is only required to adopt an average particle size obtained by measurement by a dynamic light scattering method.

The gelatin particles carry a reagent or a drug.

The gelatin particles carrying a probe means that the probe is immobilized on a surface of the gelatin particles or taken into the gelatin particles.

Note that the amount of probe in the gelatin particles is preferably larger than the amount of probe in a surface layer portion of the gelatin particles. By reducing the amount of probe in the surface layer portion of the gelatin particles, the amount of probe exposed on a surface of the gelatin particles can be reduced. As a result, the gelatin particles are less likely to be recognized as foreign substances by cells, and can be easily taken into the cells by an activity such as endocytosis. The surface layer portion means a region up to a depth of 1% with respect to the average particle size of the gelatin particles.

The reagent only needs to be a probe used for applications such as examination of activity and the like of a living body, measurement of a substance in a living body, and quantification of a substance in a living body, a contrast medium for detecting presence of cells, or the like. A detection target of the probe is not particularly limited, and may be a protein, a sugar, or a nucleic acid such as DNA or mRNA, or may be a physiological state such as temperature or pH in a cell.

The probe only needs to be, for example, a compound having a site that is directly or indirectly bonded to a substance to be detected and a site that emits a detectable signal. For example, the probe may be a probe capable of being specifically bonded to mRNA to be detected by a nucleic acid having a sequence complementary to at least a part of a nucleic acid sequence of the mRNA, or may be a probe capable of being specifically bonded to a protein to be detected by an antibody. In addition, the probe may be a probe that contains a phosphor and emits fluorescence as a signal, or may be a probe that emits another signal by chemiluminescence or the like.

The type of the phosphor is not particularly limited, and may be a fluorescent dye or a semiconductor nanoparticle.

Examples of the fluorescent dye include a rhodamine-based dye molecule, a squarylium-based dye molecule, a fluorescein-based dye molecule, a coumarin-based dye molecule, an acridine-based dye molecule, a pyrene-based dye molecule, an erythrosin-based dye molecule, an eosin-based dye molecule, a cyanine-based dye molecule, an aromatic ring-based dye molecule, an oxazine-based dye molecule, a carbopyronine-based dye molecule, and a pyrromesene-based dye molecule.

Examples of a semiconductor constituting the semiconductor nanoparticle include a group II-VI compound semiconductor, a group III-V compound semiconductor, and a group IV semiconductor. Specific examples of the semiconductor constituting the semiconductor nanoparticle include CdSe, CdS, CdTe, ZnSe, ZnS, ZnTe, InP, InN, InAs, InGaP, GaP, GaAs, Si, and Ge.

The probe capable of being specifically bonded to the mRNA may be a known probe such as a molecular beacon, a Tagman probe, a cycling probe, or an INAF probe, but a molecular beacon is preferable because the molecular beacon can use a general-purpose fluorescent dye and easily detects various cell types.

The molecular beacon is a nucleic acid derivative having a stem-loop structure, in which a fluorescent dye is bonded to one of a 5′ end and a 3′ end, and a quenching dye is bonded to the other end. In the molecular beacon, the fluorescent dye and the quenching dye are close to each other in a state where the molecular beacon forms the stem-loop structure, and thus fluorescence emitted from the fluorescent dye is quenched. However, when the molecular beacon is close to a target sequence, the loop structure is opened, and the molecular beacon is boded to mRNA to be detected. As a result, the fluorescent dye and the quenching dye are separated from each other, and fluorescence emission is detected.

A combination of the fluorescent dye and the quenching dye is not particularly limited, and only needs to be appropriately selected from the fluorescent dyes described above. The quenching dye may be a molecule that performs quenching by any of fluorescence resonance energy transfer (FRET), contact quenching, and collisional quenching.

The probe capable of being specifically bonded to a protein to be detected by the antibody is preferably a phosphor integrated Dot (PID). The PID is a nano-sized particle containing a plurality of phosphors using an organic or inorganic particle as a base body. The PID is specifically and directly or indirectly bonded to a protein to be detected by the antibody to label the protein to be detected. The plurality of phosphors may be present in the particle or on a surface of the particle. The PID can emit fluorescence having an intensity sufficient to indicate each molecule of a target substance as a bright spot.

Specific examples of the organic substance to be a material of the base body include: a thermosetting resin such as a melamine resin, a urea resin, an aniline resin, a guanamine resin, a phenol resin, a xylene resin, or a furan resin; a thermoplastic resin including a styrene resin, an acrylic resin, an acrylonitrile resin, an acrylonitrile-styrene copolymer (AS resin), and an acrylonitrile-styrene-methyl acrylate copolymer (ASA resin); other resins such as polylactic acid; and a polysaccharide. Examples of the inorganic substance to be a material of the base body include silica and glass. The base body and the fluorescent substance preferably have substituents or sites having charges opposite to each other, and have electrostatic interaction.

The average particle size of the PIDs is not particularly limited, but is preferably 10 nm or more and 500 nm or less, and more preferably 50 nm or more and 200 nm or less in consideration of ease of detection as a bright spot.

Note that the particle size of the PID can be measured by measuring a projected area of the PID using a scanning electron microscope (SEM) and converting the projected area into an equivalent circle diameter. An average particle size and a coefficient of variation of an assembly including a plurality of PIDs are calculated using a particle size (equivalent circle diameter) calculated for a sufficient number (for example, 1000) of PIDs.

The drug only needs to be able to carry gelatin particles. Examples of such a drug include: a protein having pharmaceutical activity; a nucleic acid used in pharmaceutical applications, including a plasmid, an aptamer, an antisense nucleic acid, a ribozyme, tRNA, snRNA, siRNA, shRNA, ncRNAs, and condensed DNA; and an antigen used in pharmaceutical applications.

Examples of the protein having pharmaceutical activity include a steroid, a non-steroidal anti-inflammatory drug (NSAID), vitamin A (retinoid), vitamin D3 and a vitamin D3 analog, an antibiotic, an antiviral drug, and an antibacterial drug.

The drug may be a water-soluble drug or a water-insoluble drug. Examples of the water-insoluble drug include an immunosuppressive agent such as a cyclosporine including cyclosporine, an immunoactive agent such as rapamycin, an anticancer agent such as paclitaxel, an antiviral or antibacterial agent, an anti-neoplastic tissue agent, an analgesic and an anti-inflammatory agent, an antibiotic, an anti-epileptic agent, an anxiolytic agent, an anti-anesthetic, an antagonist, a neuron blocking agent, an anti-cholinergic agent, an anti-arrhythmic agent, an anti-hypertensive agent, a hormonal agent, and a nutritional agent.

As these water-insoluble drugs, rapamycin, paclitaxel, docetaxel, and everolimus are preferable. Note that rapamycin, paclitaxel, docetaxel, and everolimus each include analogs and derivatives thereof as long as the analogs and derivatives have similar drug efficacy. For example, paclitaxel and docetaxel are in an analog relationship, and rapamycin and everolimus are in a derivative relationship. Among these, paclitaxel is more preferable.

The base material may be pre-coated with the reagent or the drug in addition to the reagent or the drug being carried by the gelatin particles. However, when the base material is coated with the reagent or the drug and the reagent or the drug is taken into cells, ease of uptake of the reagent or the drug into cells is likely to vary. Therefore, a carrying amount (mass basis) on the gelatin particles is preferably larger than a coating amount on the base material, and it is more preferable not to coat the base material with the reagent or the drug.

(Surface Charge)

According to findings of the present inventors, when the sum of charges on a surface of a cell support including the biocompatible substance and gelatin particles carried by the biocompatible substance, to which cells are attached in contact, is positive, uptake of the gelatin particles is promoted. This is considered to be because since a surface of the cells has a negative charge, when the sum of charges on a surface to which the cells are attached in contact is positive, the cells easily approach the base material or the gelatin particles, and the cells easily take the gelatin particles thereinto.

In addition, according to findings of the present inventors, when the sum of charges on a surface with which the cells are in contact is positive, culture efficiency and observation efficiency are enhanced. This is considered to be because when the sum of charges on a surface to which the cells are attached in contact is positive, adhesion of the cells to the base material is increased.

In addition, a coulomb interaction preferably occurs between the biocompatible substance and the gelatin particles in a culture environment (wet environment). This makes it possible to suppress detachment of the gelatin particles from the base material in a culture environment even by simply imparting the gelatin particles to the biocompatible substance. In addition, when the biocompatible substance has a positive charge, the cells approach the biocompatible substance more easily, and ease of uptake of the gelatin particles by the cells and an adhesion strength of the cells to the base material can be further enhanced.

More specifically, a zeta potential of the surface to which the cells are attached in contact in a solution having a pH of 7.4 is preferably more than 0 mV and 30 mV or less. When the zeta potential is more than 0 mV, uptake of the gelatin particles is easily performed, and culture efficiency and observation efficiency are enhanced. When the zeta potential is 30 mV or less, a negative influence such as a decrease in flexibility of the base material and the gelatin particles can be suppressed. The zeta potential is more preferably 2 mV or more and 30 mV or less, and still more preferably 4 mV or more and 15 mV or less from the above viewpoint.

The zeta potential can be a value measured using a known zeta potential measuring instrument (for example, a measuring instrument using a formula described in Flotation written by Hiroyuki Mori and Yoshio Okamoto, 27, 117 1-124 (1980)).

In order to achieve the above state, in the present embodiment, the gelatin particles are preferably cationized. The gelatin particles can be cationized by a method of reducing the number of carboxyl groups in a molecule by crosslinking, surface modification with PEG-NH 2, or the like. In addition, in the present embodiment, the biocompatible substance is preferably cationized. The biocompatible substance can also be cationized by a method of reducing the number of acidic functional groups such as a carboxyl group, similarly to the cationization of the gelatin particles. Only one or both of the cationization of the gelatin particles and the cationization of the biocompatible substance may be performed. The larger the degree of cationization, the easier the uptake of the gelatin particles is, and the higher culture efficiency and observation efficiency are. Meanwhile, in order to suppress a decrease in flexibility and the like due to an excessive degree of the cationization, the cationization is preferably performed to such an extent that the zeta potential of the surface to which the cells are attached in contact is 30 mV or less.

[Cell Culture Method]

A cell culture method according to another embodiment of the present invention is a cell culture method using the above-described cell support.

Cell culture according to the present embodiment can be performed in a similar manner to a known cell culture method except for using the above-described cell support as a cell support.

Specifically, the cell culture method according to the present embodiment only needs to include a step of preparing the above-described cell support and a step of seeding cells on the cell support.

For preparation of the cell support, a cell support that has already been prepared may be prepared, or a cell support may be prepared.

The preparation of the cell support can be performed by including a step of preparing a base material containing a biocompatible substance and a step of causing the base material to hold gelatin particles.

The step of preparing a base material containing a biocompatible substance is a step of preparing a base material formed of the above-described shape and material. When the base material is a formed body formed from a biocompatible substance, it is only required to prepare the formed body. Alternatively, a biocompatible substance may be imparted to a known base material, and the base material may be coated with the biocompatible substance. The biocompatible substance can be imparted by, for example, a method of imparting a solution containing the biocompatible substance to a surface of the base material, and then drying the solution. When the base material is a porous base material or the like having a three-dimensional shape, the biocompatible substance does not need to be imparted to an inner surface of the base material, but only needs to be imparted to at least an outer surface of the base material on which cells are seeded, and may be imparted only to the outer surface of the base material on which cells are seeded.

In the step of causing the base material to hold gelatin particles, it is only required to impart a solution containing the gelatin particles to the base material containing the biocompatible substance. At this time, by heating the solution containing the gelatin particles to a temperature at which the gelatin particles are solated (about 35° C. to 45° C.) and allowing the solution to stand for about one hour, the gelatin particles can be immobilized on the base material due to adhesiveness exhibited by the solated gelatin particles, and the gelatin particles can be held by the base material. Thereafter, the solution may then be dried.

At this time, the gelatin particles are immobilized and held at positions in contact with the biocompatible substance contained in the base material. For example, when the biocompatible substance is imparted to a surface of the base material, the gelatin particles are imparted to and immobilized on a region of the base material to which the biocompatible substance is imparted. When the base material is a porous base material or the like having a three-dimensional shape, the gelatin particles do not need to be imparted to an inner surface of the base material to be immobilized, but only need to be imparted to at least an outer surface of the base material on which cells are seeded to be immobilized, and may be imparted only to the outer surface of the base material on which cells are seeded to be immobilized.

The gelatin particles carry a reagent or a drug. It is only required to cause the gelatin particles to carry a reagent or a drug by a known method of mixing the gelatin particles with the reagent or the drug, or adding the reagent or the drug to a solution used for preparing the gelatin particles.

Note that, also in this case, when the base material is a porous base material or the like having a three-dimensional shape, gelatin particles do not need to be imparted to an inner surface of the base material to be immobilized, but only need to be immobilized on at least an outer surface of the base material on which cells are seeded to be held.

In this case, the type of biocompatible substance or the type of gelatin particles may be selected such that the sum of charges of a surface to which cells of the cell support are attached in contact is positive, coulomb interaction occurs between the biocompatible substance and the gelatin particles in a culture environment (in a wet environment), or a zeta potential in a solution having a pH of 7.4 on the surface to which the cells are attached in contact is more than 0 mV and 30 mV or less. For example, the cell support may be prepared using a cationized biocompatible substance or cationized gelatin particles.

It is only required to seed the cells by a normal method.

The cells to be seeded only need to be cells desired to be cultured and stored as necessary. Examples of the cells include cells derived from a biological sample or a specimen extracted from various organs including bone marrow, heart, lung, liver, kidney, pancreas, spleen, intestinal tract, small intestine, heart valve, skin, blood vessel, cornea, eyeball, dura mater, bone, trachea, and ossicle, commercially available established cells, and known cells including stem cells including skin stem cells, epidermal keratinized stem cells, retinal stem cells, retinal epithelial stem cells, cartilage stem cells, hair follicle stem cells, muscle stem cells, bone precursor cells, fat precursor cells, hematopoietic stem cells, neural stem cells, hepatic stem cells, pancreatic stem cells, ectodermal stem cells, mesodermal stem cells, endodermal stem cells, mesenchymal stem cells, ES cells, and iPS cells, and cells differentiated from these stem cells. In addition, the cells to be seeded may be cells of an organism other than animals, such as a plant, a germ, a protist, or a bacterium.

At this time, a known medium may be imparted to the cell holder to promote growth of the cells.

The seeded cells proliferate using the cell holder as a scaffold to form a colony while taking the gelatin particles held by the cell holder thereinto by endocytosis. Then, the reagent or the drug carried by the gelatin particles is slowly released from the gelatin particles that have been taken into the cells. In this way, a colony is formed by cells containing the reagent or the drug.

Conventionally, an attempt has been made to take the reagent or the drug into cells from a solution containing the reagent or the drug, or an attempt has been made to introduce the reagent or the drug into cells by imparting a dispersion containing gelatin particles carrying the reagent or the drug to a medium such that the gelatin particles are taken into the cells. According to findings of the present inventors, when the gelatin particles held by the cell holder are taken into cells, a reagent or a drug can be uniformly introduced into more cells than as compared with a case performed by these methods.

Note that, when the base material is a porous base material or the like having a three-dimensional shape, the cells only need to be seeded on a region holding gelatin particles on a surface of the cell holder. The seeded cells take gelatin particles held by the cell holder thereinto by endocytosis. Thereafter, when the cells that have taken the gelatin particles thereinto proliferate while dividing and enter the inside of the base material, it is considered that the reagent or the drug carried by the gelatin particles is also taken over by each of the divided cells to form a colony by the cells containing the reagent or the drug.

[Cell Structure]

A cell structure according to another embodiment of the present invention is a cell structure including the above-described cell support.

The cell structure includes a base material containing the above-described biocompatible substance and a plurality of cells instructed by the cell support. The gelatin particles held by the base material and the reagent or the drug carried by the gelatin particles that have been taken into the cells.

In the cell culture body, the gelatin particles, or the reagent or the drug has been uniformly taken into more cells. For example,

the cell culture body can be used for transplantation into a living body, storage of cells, and the like. At this time, since the reagent or the drug carried by the gelatin particles has been uniformly taken into more cells, a state such as differentiation of cells, life and death of cells, and the like can be detected with higher sensitivity.

### EXAMPLES

Hereinafter, specific Example of the present invention will be described together with Comparative Example, but the present invention is not limited thereto.

### 1. Probe

The following probe was used.

GAPDH-MB: A probe in which a 5′ end of a base sequence containing a sequence complementary to mRNA of glutaraldehyde-3-phosphate dehydrogenase (GAPDH) is modified with AlexaFlour 488, and a 3′ end thereof is modified with iowa black RQ (IBRQ). GAPDH-MB is a molecular beacon in which the 5′ end site and the 3′ end site are complementary sequences constituting a stem region, and a site therebetween is a sequence constituting a loop structure.

It was previously confirmed that a fluorescence intensity from the molecular beacon emitted fluorescence only when the molecular beacon reacted with mRNA of glutaraldehyde-3-phosphate dehydrogenase (GAPDH) mRNA, which is mRNA constantly expressed in cells, and that the fluorescence intensity increased according to the amount of each mRNA.

### 2. Experiment 1

### 2-1. Preparation of Gelatin Particles

Gelatin (G-2613P manufactured by Nitta Gelatin Inc.) was dissolved in 24 ml of a 0.1 M phosphate buffer aqueous solution (pH 5.0) at 37° C. To this solution, an appropriate amount of ethylenediamine was added. Furthermore, a hydrochloric acid aqueous solution was added thereto to adjust the pH of the solution to 5.0. Furthermore, an appropriate amount of 1-ethyl-3-(3-dimethylaminopropyl) carbodiimide hydrochloride was added thereto, and the concentration of gelatin was adjusted to 2% by mass by addition of a 0.1 M phosphate buffer aqueous solution. This solution was stirred at 37° C. for four hours to introduce ethylenediamine into a carboxyl group of the gelatin. Thereafter, the reaction product was dialyzed in redistilled water for three days to obtain cationized gelatin in a slurry state. Thereafter, acetone as a phase separation inducer was added thereto and mixed at 50° C., and the particles precipitated in the slurry were collected and washed with pure water to obtain cationized gelatin particles. The cationized gelatin particles are referred to as cGNS.

An apparent average particle size of cGNS was determined by a dynamic light scattering method at 37° C. using DLS-7000 manufactured by Otsuka Electronics Co., Ltd., and found to be 168.0 nm. A zeta potential of cGNS was determined by an electrophoretic light scattering method using DLS-8000 manufactured by Otsuka Electronics Co., Ltd., and found to be 8.41 mV.

### 2-2. Carrying of Molecular Beacon by Gelatin Particles

cGNS and GAPDH-MB were mixed at room temperature for 15 minutes, and then centrifuged and washed with water to obtain gelatin particles carrying the probe. The gelatin particles are referred to as cGNS (GAPDH-MB).

### 2-3. Carrying of Gelatin Particles by Base Material

As a base material having a two-dimensional shape, a cell culture plate (96-well Edge Plate manufactured by Thermo Fisher Scientific Inc.) was prepared. The cell culture plate was coated with gelatin (Type B, isoelectric point 5, manufactured by Nitta Gelatin Inc.).

cGNS (GAPDH-MB) was added to the base material, and the resulting mixture was allowed to stand at 37° C. for one hour to immobilize cGNS (GAPDH-MB), thereby obtaining a cell support.

### 2-4. Cell Culture on Cell Support

MC3T3-E1 cells which are mouse mesenchymal stem cell lines were seeded on the cell support, and the cells were cultured for one day.

### 2-5. Observation

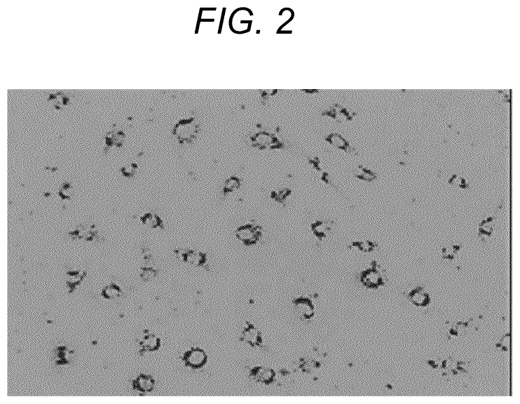

FIG. 2 is a fluorescence image observed one day after seeding. As is clear from FIG. 2, red fluorescence was uniformly observed from the entire plate. This is considered to be because GAPDH-MB was uniformly taken into the entire cells grown on the cell support, and reacted with mRNA of GAPDH in the cells to emit red fluorescence.

### 3. Experiment 2

### 3-1. Preparation of Gelatin Particles and Carrying of Molecular Beacon by Gelatin Particles

As in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 3-2. Carrying of Gelatin Particles by Base Material

Gelatin hydrogel particles were dispersed in PBS to prepare swollen and thermally crosslinked gelatin hydrogel particles (scaffold material for spheroid formation). The thermally crosslinked gelatin hydrogel particles had a particle size of 32 to 53 mm. The thermally crosslinked gelatin hydrogel particles and the gelatin particles cGNS (GAPDH-MB) were mixed, and the resulting mixture was allowed to stand at 37° C. for one hour to immobilize cGNS (GAPDH-MB) on the thermally crosslinked gelatin hydrogel particles, thereby obtaining a cell support.

### 3-3. Seeding of Cells on Cell Support

The cell support and MC3T3-E1 cells which are mouse mesenchymal stem cell lines were introduced into a polyvinyl alcohol-coated U-bottomed 96-well plate, and the cells were cultured for three days to form a cell aggregate.

### 3-4. Observation

Three days after seeding, the cell aggregate was immobilized with 4% paraformaldehyde to prepare a frozen section of a center plane of the cell aggregate. To this frozen section, 4′,6-diamidino-2-phenylindole (DAPI) was added to stain a nucleus, and then the nucleus was observed with a fluorescence microscope.

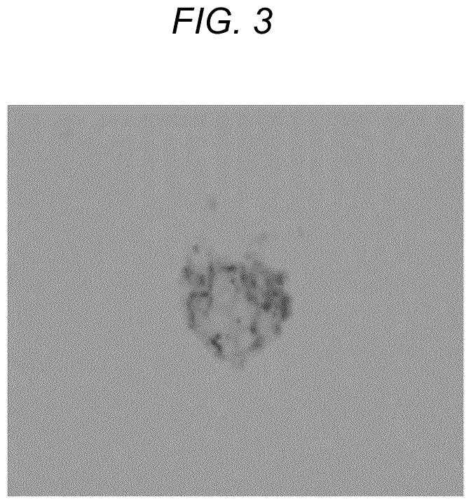

FIG. 3 is a fluorescence image observed at this time. As is clear from FIG. 3, red fluorescence was uniformly observed from the entire plate. This is considered to be because GAPDH-MB was uniformly taken into the entire cells grown on the cell aggregate, and reacted with mRNA of GAPDH in the cells to emit red fluorescence.

### 4. Experiment 3

### 4-1. Preparation of Gelatin Particles

As in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 4-2. Carrying of Gelatin Particles by Base Material

Non-thermally crosslinked gelatin nonwoven fabric 1 (Genocel (“Genocel” is a registered trademark of THE JAPAN WOOL TEXTILE.CO., LTD.) manufactured by THE JAPAN WOOL TEXTILE.CO., LTD.) was prepared. cGNS (GAPDH-MB) was added to the base material, and the resulting mixture was allowed to stand at 37° C. for one hour to immobilize cGNS (GAPDH-MB), thereby obtaining a cell support.

### 4-3. Cell Culture

MC3T3-E1 cells which are mouse mesenchymal stem cell lines were seeded on the cell support, and the cells were cultured for one day. As Comparative Example, MC3T3-E1 cells were seeded on a 6-well plate, and the cells were cultured for one day in the presence of an OptiMEM culture solution to which cGNS (GAPDH-MB) had been added.

### 4-4. Observation

One day after seeding, the cultured cells were observed with a fluorescence microscope, and an imaging fluorescence intensity of each of six randomly imaged visual fields was measured. The imaging fluorescence intensity was divided by the number of cells confirmed in each of the visual fields to calculate an average fluorescence intensity per number of cells, and a standard deviation for each of the visual fields was also calculated.

Table 1 presents the average fluorescence intensity per number of cells and the standard deviation.

TABLE 1

| | | Average fluorescence | Standard |
|---|---|---|---|
| | Gelatin particles | intensity per cell | deviation |
| Example | Immobilized on base | 16.8 | 2.6 |
| | material (cell support) | | |
| Comparative | Added to culture | 12.1 | 5.6 |
| Example | solution | | |

As presented in Table 1, when the cell support was used, the average fluorescence intensity per cell was higher and the standard deviation was smaller. From this result, it has been found that when a cell support including a base material containing a biocompatible substance and gelatin particles held by the base material is used, uptake of a molecular beacon (drug or reagent) by cells is promoted, and the molecular beacon is taken into the cells more uniformly.

### 5. Experiment 4

### 5-1. Preparation of Gelatin Particles

As in Experiment 1, cationized gelatin particles cGNS were prepared. At this time, three kinds of cGNS (cGNS1 to cGNS3) having different average particle sizes were adjusted by changing the preparation conditions. Each of cGNS1 to cGNS3 and GAPDH-MB were mixed at room temperature for 15 minutes, and then centrifuged and washed with water to obtain gelatin particles cGNS1 (GAPDH-MB) to cGNS3(GAPDH-MB) carrying the probe.

### 5-2. Carrying of Gelatin Particles by Base Material

A non-thermally crosslinked gelatin nonwoven fabric (Genocel manufactured by THE JAPAN WOOL TEXTILE.CO., LTD.) was prepared. Each of cGNS1 (GAPDH-MB) to cGNS3(GAPDH-MB) was added to the base material, and the resulting mixture was allowed to stand at 37° C. for one hour to immobilize cGNS1 (GAPDH-MB) to cGNS3(GAPDH-MB), thereby obtaining cell supports 1 to 3.

### 5-3. Cell Culture

MC3T3-E1 cells which are mouse mesenchymal stem cell lines were seeded on each of cell supports 1 to 3, and the cells were cultured for one day.

### 5-4. Observation

One day after seeding, the cultured cells were observed with a fluorescence microscope, and an imaging fluorescence intensity of each of six randomly imaged visual fields was measured. The imaging fluorescence intensity was divided by the number of cells confirmed in each of the visual fields to calculate an average fluorescence intensity per number of cells.

Table 2 presents the average fluorescence intensity per number of cells.

TABLE 2

| Average particle size of | Average fluorescence |
|---|---|
| gelatin particles (nm) | intensity per cell |
| 25 | 2.5 |
| 50 | 4.3 |
| 500 | 21.3 |

As presented in Table 2, it has been found that the larger the average particle size of the gelatin particles, the more easily the gelatin particles and the molecular beacon (drug or reagent) carried by the gelatin particles are taken into cells by the cells' own activity.

### 6. Experiment 5

### 6-1. Preparation of Gelatin Particles

As in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 6-2. Carrying of Gelatin Particles by Base Material

As a base material having a three-dimensional shape, non-thermally crosslinked gelatin nonwoven fabric 1 (Genocel manufactured by THE JAPAN WOOL TEXTILE.CO., LTD.), thermally crosslinked gelatin nonwoven fabric 2 (Genocel manufactured by THE JAPAN WOOL TEXTILE.CO., LTD., heat treatment time: 4 to 24 hours), and a polypropylene nonwoven fabric (polypropylene long fiber nonwoven fabric manufactured by Toray Industries, Inc.) were prepared. Note that, for the polypropylene nonwoven fabric, a base material coated with gelatin and a base material not coated with gelatin were prepared.

cGNS (GAPDH-MB) was added to each of these base materials, and the resulting mixture was allowed to stand at 37° C. for one hour to immobilize cGNS (GAPDH-MB), thereby obtaining a cell support.

### 6-3. Seeding of Cells on Cell Support

MC3T3-E1 cells which are mouse mesenchymal stem cell lines were seeded on the cell support, and the cells were cultured for one day.

### 6-4. Observation

One day after seeding, PBS containing CYTO 13 was added to the cell support to stain a nucleus, and the resulting mixture was allowed to stand at 37° C. for one hour. Thereafter, each cell support was imaged on a glass bottom dish and observed with a confocal laser microscope.

FIG. 4A is a fluorescence image observed from a cell support using gelatin nonwoven fabric 1. FIG. 4B is a fluorescence image observed from a cell support using gelatin nonwoven fabric 2. FIG. 4C is a fluorescence image observed from a cell support using a polypropylene nonwoven fabric coated with gelatin. FIG. 4D is a fluorescence image observed from a cell support using a polypropylene nonwoven fabric not coated with gelatin.

As is clear from FIGS. 4A to 4C, red fluorescence was uniformly observed from the entire plate. This is considered to be because GAPDH-MB was uniformly taken into the entire cells grown on the cell aggregate, and reacted with mRNA of GAPDH in the cells to emit red fluorescence. In addition, the fluorescence intensity varied depending on the type of base material. From this result, it has been found that the introduction ratio of GAPDH-MB varies depending on the state of the base material, and it has been found that the introduction ratio of GAPDH-MB can be changed by changing the state of the base material.

Meanwhile, as is clear from FIG. 4D, when cGNS (GAPDH-MB) was immobilized on a base material to which a biocompatible substance had not been imparted, and cells were cultured, red fluorescence was not observed. From this result, it has been found that uptake of cGNS (GAPDH-MB) is promoted when the base material contains a biocompatible substance.

### 7. Experiment 6

### 7-1. Preparation of Gelatin Particles

As in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 7-2. Carrying of Gelatin Particles by Base Material

As a base material, a polypropylene (PP) well plate, a polypropylene (PP) nonwoven fabric (polypropylene long fiber nonwoven fabric manufactured by Toray Industries, Inc.), a well plate coated with polyvinyl alcohol (PVA), a well plate coated with gelatin, and a gelatin nonwoven fabric (Genocel manufactured by THE JAPAN WOOL TEXTILE.CO., LTD.) were prepared. cGNS (GAPDH-MB) was added to each of these base materials, and the resulting mixture was allowed to stand at 37° C. for one hour to immobilize cGNS1 (GAPDH-MB), thereby obtaining cell supports 1 to 5.

### 7-3. Cell Culture

MC3T3-E1 cells which are mouse mesenchymal stem cell lines were seeded on each of the cell supports 1 to 5, and the cells were cultured for one day.

### 7-4. Observation

One day after seeding, the cultured cells were observed with a fluorescence microscope, and an imaging fluorescence intensity of each of six randomly imaged visual fields was measured. The imaging fluorescence intensity was divided by the number of cells confirmed in each of the visual fields to calculate an average fluorescence intensity per number of cells.

Table 3 presents the average fluorescence intensity per number of cells.

TABLE 3

| | | Average fluorescence |
|---|---|---|
| Cell support | Material of base material | intensity per cell |
| 1 | PP | 0.5 |
| 2 | PP nonwoven fabric | 0.7 |
| 3 | PVA coating | 13.8 |
| 4 | Gelatin coating | 16.7 |
| 5 | Gelatin nonwoven fabric | 21.3 |

As presented in Table 3, when the base material contained a biocompatible substance, the average fluorescence intensity per cell was high. From this result, it has been found that uptake of cGNS (GAPDH-MB) is promoted when the base material contains a biocompatible substance.

### 8-1. Experiment 7

As in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 8-2. Carrying of Gelatin Particles by Base Material

As a base material, a non-thermally crosslinked gelatin nonwoven fabric (Genocel manufactured by THE JAPAN WOOL TEXTILE.CO., LTD.), thermally crosslinked gelatin nonwoven fabric 2 (Genocel manufactured by THE JAPAN WOOL TEXTILE.CO., LTD., heat treatment time: 4 hours), and thermally crosslinked gelatin nonwoven fabric 3 (Genocel manufactured by THE JAPAN WOOL TEXTILE.CO., LTD., heat treatment time: 24 hours) were prepared. In addition, gelatin nonwoven fabric 4 obtained by cationizing a non-thermally crosslinked gelatin nonwoven fabric (Genocel manufactured by THE JAPAN WOOL TEXTILE.CO., LTD.) by introduction of an amino group, and gelatin nonwoven fabric 5 obtained by anionizing a non-thermally crosslinked gelatin nonwoven fabric (Genocel manufactured by THE JAPAN WOOL TEXTILE.CO., LTD.) by introduction of succinic anhydride into an amino group of gelatin were prepared. cGNS (GAPDH-MB) was added to each of these base materials, and the resulting mixture was allowed to stand at 37° C. for one hour to immobilize cGNS1 (GAPDH-MB), thereby obtaining cell supports 5 to 9. Note that cell support 5 is the same as cell support 5 in Experiment 6.

A zeta potential of each of cell supports 5 to 7 in a solution having a pH of 7.4 was measured with a nanoparticle analyzer nanoPartica SZ-100V2 (product name) manufactured by HORIBA, Ltd.

### 8-3. Cell Culture

MC3T3-E1 cells which are mouse mesenchymal stem cell lines were seeded on each of the cell supports 1 to 5, and the cells were cultured for one day.

### 8-4. Observation

One day after seeding, the cultured cells were observed with a fluorescence microscope, and an imaging fluorescence intensity of each of six randomly imaged visual fields was measured. The imaging fluorescence intensity was divided by the number of cells confirmed in each of the visual fields to calculate an average fluorescence intensity per number of cells.

Table 4 presents the zeta potential of each of the cell supports and the average fluorescence intensity per number of cells.

TABLE 4

| | | Zeta | |
|---|---|---|---|
| Cell | | potential | Average fluorescence |
| support | Material of base material | (mV) | intensity per cell |
| 5 | Gelatin nonwoven fabric | 2.8 | 21.3 |
| | (non-thermally crosslinked) | | |
| 6 | Gelatin nonwoven fabric | 4.5 | 25.4 |
| | (thermally crosslinked | | |
| | (four hours)) | | |
| 7 | Gelatin nonwoven fabric | 28.3 | 12.8 |
| | (thermally crosslinked | | |
| | (24 hours)) | | |
| 8 | Gelatin nonwoven fabric | 10.2 | 31.2 |
| | (cationized) | | |
| 9 | Gelatin nonwoven fabric | −12.6 | 2.3 |
| | (anionized) | | |

As presented in Table 4, it has been found that uptake of gelatin particles is promoted by anionizing the biocompatible substance contained in the base material and adjusting the zeta potential of the cell support.

### 9. Experiment 8

### 9-1. Preparation of Gelatin Particles

As in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 9-2. Preparation of Base Material

A non-thermally crosslinked gelatin nonwoven fabric (Genocel manufactured by THE JAPAN WOOL TEXTILE.CO., LTD.) was prepared. cGNS (GAPDH-MB) was added to the base material, and the resulting mixture was allowed to stand at 37° C. for one hour to immobilize cGNS (GAPDH-MB), thereby obtaining a cell support.

As Comparative Example, a drug (paclitaxel) dissolved in a solvent (anhydrous ethanol) was applied to a base material (gelatin nonwoven fabric (Genocel manufactured by THE JAPAN WOOL TEXTILE.CO., LTD.)), and dried to prepare a base material coated with the drug.

### 9-3. Cell Culture

SK-BR-3 cells which are human breast cancer cell lines were seeded on each of the cell support and the base material coated with the drug, prepared above, and the cells were cultured for 24 to 48 hours.

### 9-4. Observation

A cell aggregate was immobilized with 4% paraformaldehyde to prepare a frozen section of a center plane of the cell aggregate. To this frozen section, 4′,6-diamidino-2-phenylindole (DAPI) was added to stain a nucleus, then the nucleus was observed with a fluorescence microscope, and an imaging fluorescence intensity of each of six randomly imaged visual fields was measured. The imaging fluorescence intensity was divided by the number of cells confirmed in each of the visual fields to calculate an average fluorescence intensity per number of cells, and a standard deviation for each of the visual fields was also calculated.

Table 5 presents the average fluorescence intensity per number of cells and the standard deviation.

TABLE 5

| | | Average fluorescence | Standard |
|---|---|---|---|
| | Drug | intensity per cell | deviation |
| Example | Gelatin particles | 18.9 | 1.8 |
| | carrying drug are | | |
| | immobilized on base | | |
| | material (cell support) | | |
| Comparative | Base material is coated | 30.8 | 5.8 |
| Example | with drug | | |

As presented in Table 5, when the cell support was used, the standard deviation was smaller. Meanwhile, when the base material is only coated with the drug, it has been found that the drug is sufficiently taken into cells from the average fluorescence intensity per cell. However, meanwhile, it has been found that the standard deviation is large, and the uptake of the drug into the cells varies. From this result, it has been found that when a cell support including a base material containing a biocompatible substance and gelatin particles held by the base material is used, a drug or a reagent is taken into cells more uniformly.

The present application claims a priority based on International Application No. PCT/JP2020/035197 filed on Sep. 17, 2020, and the contents described in the claims, the description, and the drawings of the application are incorporated into the present application.

### INDUSTRIAL APPLICABILITY

According to the present invention, it is possible to obtain a cell structure in which a reagent or a drug has been uniformly taken into more cells. This cell structure can be suitably used for transplantation and storage of cells.

### REFERENCE SIGNS LIST

100 Cell support
112 Base material containing biocompatible substance
114 Base material such as glass or plastic
116 Biocompatible substance
120 Gelatin particle
130 Cell

1. A cell support comprising:
a base material containing a biocompatible substance or a base material having a surface to which the biocompatible substance is imparted; and
gelatin particles held by the biocompatible substance of the base material.

2. The cell support according to claim 1, wherein the base material is a molded body of a biocompatible substance.

3. The cell support according to claim 1, wherein the base material includes a molded body and a biocompatible substance imparted to a surface of the molded body.

4. The cell support according to claim 1, wherein the biocompatible substance is a polymer derived from a living body or a biodegradable synthetic polymer.

5. The cell support according to claim 4, wherein the biocompatible substance is at least one biocompatible substance selected from the group consisting of collagen, gelatin, fibronectin, polyvinyl alcohol, and a copolymer thereof.

6. The cell support according to claim 1, wherein in the biocompatible substance and the gelatin particles, a Coulomb interaction occurs between the biocompatible substance and the gelatin particles in a culture environment.

7. The cell support according to claim 1, wherein a sum of charges on a surface to which cells are attached is positive.

8. The cell support according to claim 1, wherein a zeta potential of a surface to which cells are attached in a solution having a pH of 7.4 is more than 0 mV and 30 mV or less.

9. The cell support according to claim 1, wherein a zeta potential of a surface to which cells are attached in a solution having a pH of 7.4 is 4 mV or more and 15 mV or less.

10. The cell support according to claim 1, wherein the biocompatible substance is cationized.

11. The cell support according to claim 1, wherein the gelatin particles are cationized.

12. The cell support according to claim 1, wherein the biocompatible substance is a material selected according to an introduction ratio of the gelatin particles to be achieved for cells supported by the cell support.

13. The cell support according to claim 1, wherein the base material has a two-dimensional shape.

14. The cell support according to claim 1, wherein the base material has a three-dimensional shape.

15. The cell support according to claim 1, wherein the gelatin particles carry a reagent or a drug.

16. The cell support according to claim 15, wherein the reagent or the drug is a molecular beacon.

17. A method of producing a cell support, comprising:
preparing a base material containing a biocompatible substance; and
imparting gelatin particles to the base material to cause the base material to hold the gelatin particles.

18. The method of producing a cell support according to claim 17, wherein selection of the base material or selection of the gelatin particles causes a Coulomb interaction between the biocompatible substance and the gelatin particles in a culture environment.

19. The method of producing a cell support according to claim 17, wherein selection of the base material or selection of the gelatin particles makes a sum of charges on a surface to which cells are attached positive.

20. The method of producing a cell support according to claim 17, wherein selection of the base material or selection of the gelatin particles makes a zeta potential of a surface to which cells are attached more than 0 mV and 30 mV or less.

21. The method of producing a cell support according to claim 17, wherein selection of the base material or selection of the gelatin particles makes a zeta potential of a surface to which cells are attached 4 mV or more and 15 mV or less.

22. The method of producing a cell support according to claim 17, wherein the base material contains the biocompatible substance that has been cationized.

23. The method of producing a cell support according to claim 17, wherein the gelatin particles are cationized.

24. The method of producing a cell support according to claim 17, wherein the biocompatible substance is a material selected according to an introduction ratio of the gelatin particles to be achieved for cells supported by the cell support.

25. A cell culture method comprising:
preparing the cell support according to claim 1; and
seeding cells on the cell support.

26. A cell structure comprising:
the cell support according to claim 1; and
cells held by the cell support.
